(19) United States
(12) Patent Application Publication
(10) Pub. No.: US 2019/0038336 A1
(43) Pub. Date: Feb. 7, 2019

(54) ELECTROSURGICAL FORCEPS

(71) Applicant: Elliquence, Baldwin, NY (US)

(72) Inventors: Alan Ellman, Hewlett, NY (US); Spencer Ellman, New York, NY (US)

(21) Appl. No.: 16/055,581

(22) Filed: Aug. 6, 2018

Related U.S. Application Data
(60) Provisional application No. 62/542,226, filed on Aug. 7, 2017.

(51) Int. Cl. A61B 18/12; A61B 17/29; A61B 18/14

(52) U.S. Cl. CPC A61B 18/1206; A61B 17/29; A61B 2018/00083; A61B 2018/126; A61B 18/1442

(57) ABSTRACT

Electrosurgical forceps have a body portion and two buttons. A first electrode is supported by the body portion and terminates at a first end. A second electrode is positioned parallel to the first electrode. The second electrode terminates at a second end. A sheath is positioned around the first electrode and the second electrode and terminates at a sheath end. The first end and the second end extend axially out from the sheath end, wherein the sheath is movably supported by the body portion.

### PRIORITY

The present application claims priority and incorporates by reference U.S. Provisional Patent Application 62/542,226 entitled Electrosurgical Forceps.

### SUMMARY

The present invention relates to electrosurgical forceps, and more specifically, the present invention relates to a electrosurgical forceps that provide enhanced grasping during surgery.

### BACKGROUND

In the field of surgery, there is a need for a surgeon to undertake dutiful and extreme care when grasping materials in the operative field. This is particularly true during neurosurgery, where the surgeon often has to move nerves or other sensitive materials. In spinal surgery, such grasping may also be associated with repairing herniated discs.

In one example, electrosurgical forceps are employed to grasp materials in the back and provide electrosurgical current to those materials. Here, the surgeon is required to exercise extreme care in that only the desired materials are grasped as, after grasping, electrosurgical current is applied thereto. Failure to accurately grasp only the right materials may result in damage to the surrounding nerves and patient particularly when electrosurgical current is employed.

In the present state of the art, certain drawbacks exist relating to this accuracy. Movement of the forceps may accompany their actuation or movement of the surgeons hands may inhibit accurate grasping.

### SUMMARY

Electrosurgical forceps have a body portion and two buttons. A first electrode is supported by the body portion and terminates at a first end. A second electrode is positioned parallel to the first electrode. The second electrode terminates at a second end.

A sheath is positioned around the first electrode and the second electrode and terminates at a sheath end. The first end and the second end extend axially out from the sheath end, wherein the sheath is movably supported by the body portion.

A beveled bushing is positioned in an end cap and is axially pressed against chamfered regions on the first button and the second button. The beveled bushing is rigidly connected to the sheath. The first end and the second end are spaced when the first button and the second button are un-pressed. The sheath moves over the first end and the second end to press the first end against the second end when the buttons are pressed.

### BRIEF DESCRIPTION OF THE DRAWINGS

FIG. 1 is a perspective view of electrosurgical forceps according to one aspect of the invention;

FIG. 2 is a side view of electrosurgical forceps according to one aspect of the invention;

FIG. 3 is a magnified view of III-III in FIG. 2;

FIG. 4 is a cross-sectional view of electrosurgical forceps according to one aspect of the invention;

FIG. 5 is a perspective view of electrosurgical forceps during usage according to one aspect of the invention; and

FIG. 6 is a perspective view of electrosurgical forceps during usage according to one aspect of the invention.

### DETAILED DESCRIPTION OF PREFERRED EMBODIMENTS

The present application incorporates U.S. Pat. No. 7,137,982, 7,101,370 and 8,409,194 and patent application Ser. No. 15/151,422 entirely by reference.

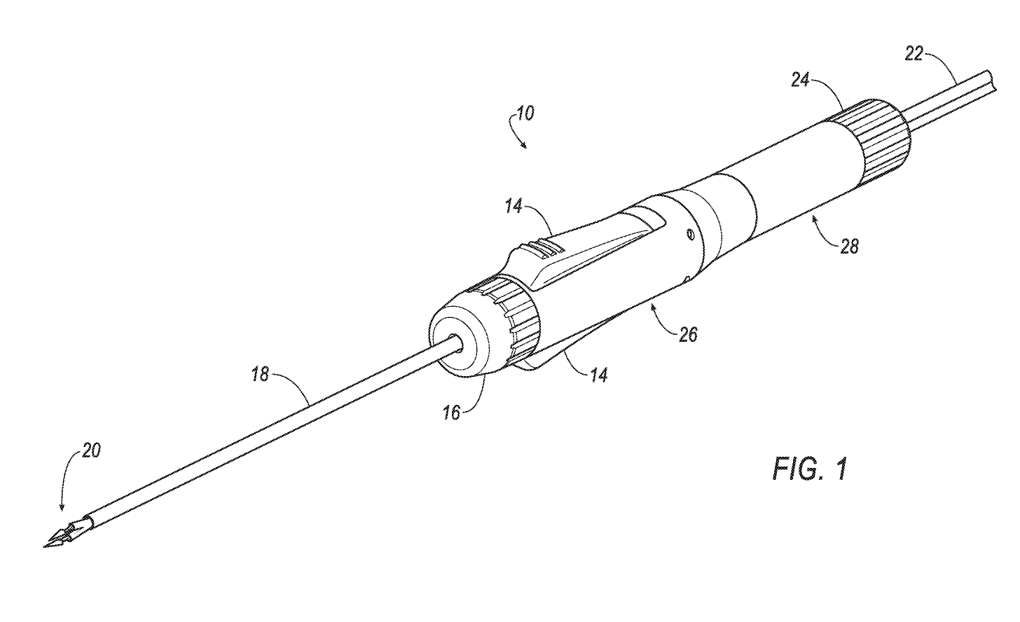

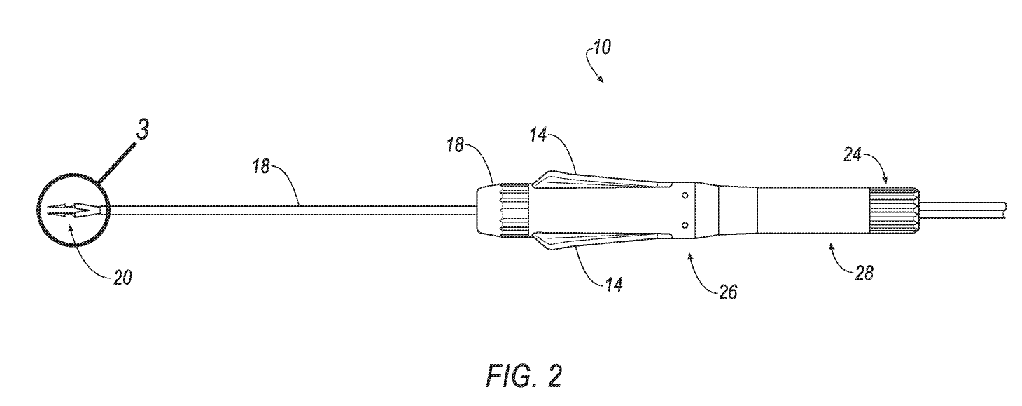

Referring now to FIGS. 1 and 2, an electrosurgical forceps 10 is shown and described. In FIGS. 1 and 2, electrosurgical forceps 10 generally includes an activator portion 26 that includes the mechanism for actuating the instrument and a body portion 28 that includes the electrical connections to an RF generator. As shown in the FIG., activator portion 26 and body portion 28 form a generally cylindrical tool for containing the internal components and for ease of being held by a surgeon. An electric cable 22 enters the body portion from an RF generator to provide electrosurgical current for the electrosurgical forceps 10. End cap 16 is located at an opposite end of the activator portion 26 with respect to the body portion 28 and contains various components for actuation of the instrument as will be described in greater detail. It will be understood that other configurations may also be employed by the instrument.

Sheath 18 extends axially from the activator portion 26 and contains an electrode that passes electrosurgical current from electric cable 22 to forceps 20. Buttons 14 are positioned on opposite radial side of and extend through the outer surface of activator portion 26. As will be discussed, buttons 14, when actuated, engage components inside the actuator portion 26 to move sheath 18 to the left in the FIG (or axially with respect to the device) when the buttons are pressed and released.

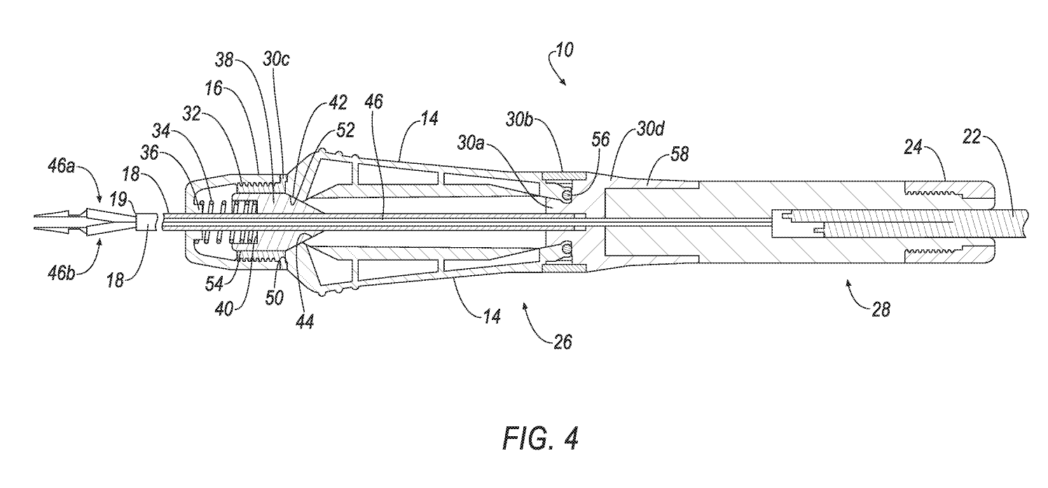

With reference to FIG. 4, a cross-sectional view of FIGS. 1 and 2 of the electrosurgical forceps 10 is provided to describe one embodiment of the mechanics inside the electrosurgical forceps 10. Here, activator portion 26 includes support structure areas that support the buttons 14, sheath 18 and other components as will be described. The support structure, comprising areas 30a, 30b, 30c and 30d, provides a framework on which various components are located and mounted. For example, sheath 18 is in sliding engagement with an inner diameter of support structure 30a and end cap 16 is threateningly engaged to support structure 30c.

In FIG. 4, buttons 14 are shown being connected to the body portion 28 and rotatably mounted about hinges 56. Hinges 56 are, in turn, mounted to the support structure 30b. Hinges 56 allow the buttons to be pressed inward toward the axial center of the electrosurgical forceps 10 such that the end proximate the hinge acts as a center of rotation while the distal end moves to actuate the internal components.

Inside the end cap 16 are a number of components that move in response to movement of the buttons to actuate the device. For example, sheath bushing 38 is provided inside the end cap and has a beveled face 42 that abuts against chamfered region 52 of each of the buttons 14. Accordingly, when the buttons 14 are pressed, they rotate about the hinge 56 to press chamfered region 52 against beveled face 42.

Sheath bushing 38 is generally supported by the inside diameter of support structure 30c, which is in sliding engagement with the outer diameter of the sheath bushing 38, to permit sheath bushing 38 to move back and forth in the axial direction with respect to the FIG. A spring 34 is located on a locating barrel 36 of the end cap 16. At an opposite end, the spring 34 is positioned in recess 40 within the sheath bushing 38. In this way, the spring is restricted from radial or axial movement and acts to bias the sheath bushing 38 against the chamfered region 52.

With continued reference to FIG. 4, body portion 28 contains the electrical connections to connect the electrosurgical device 10 to an RF generator (see the '370 patent for an example of the connection). Here, electric cable 22 passes into body portion 28 to supply RF current to the electrosurgical forceps 10. Electrode 46 is supported inside the support structure 30d. In one aspect, electrode 46 is a bipolar electrode that extends out of sheath 18 and terminates at forceps 20 (see FIG. 1).

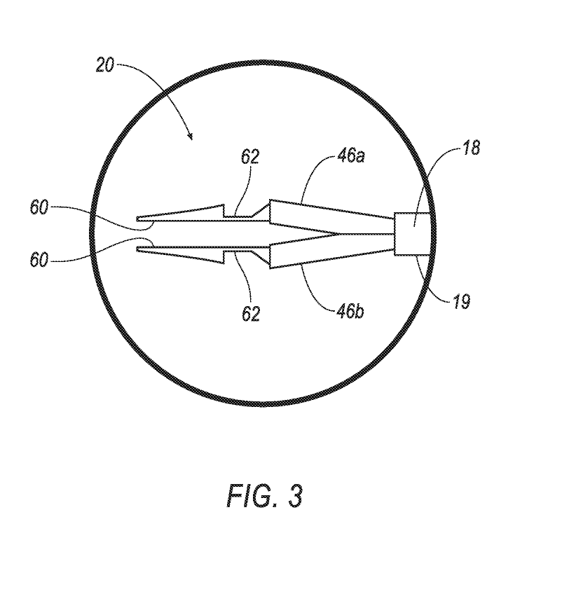

Referring now to FIG. 3, a magnification of view 3 in FIG. 2 is shown in more detail. Here, forceps 26 are shown extending from electrodes 46a and 46b. More specifically, electrode 46 is a bipolar electrode comprising two electrodes: 46a and 46b that expand out from sheath 18 to form a V-shape. The electrodes are spring-like biased to expand such that, when not within sheath 18, they naturally expand to the configuration shown. Connections 62 then connect the electrodes to respective surface regions 60 that will be described in more detail.

Referring now to FIGS. 3, 4 and 5, one example of the operation of the present invention will be described. In FIG. 4, the electrosurgical forceps 10 is shown in an unactuated state. Here, buttons 14 are not depressed and, as such, spring 34 retains sheath bushing 38 in the most right-hand position. Accordingly, sheath 18 is also positioned in the most right-hand location with respect to FIG. 4, thereby maintaining sheath 19 away from forceps 20. The elastic nature of forceps 20, particularly electrodes 46, are naturally sprung such that they are spread apart when the sheath does not surround them.

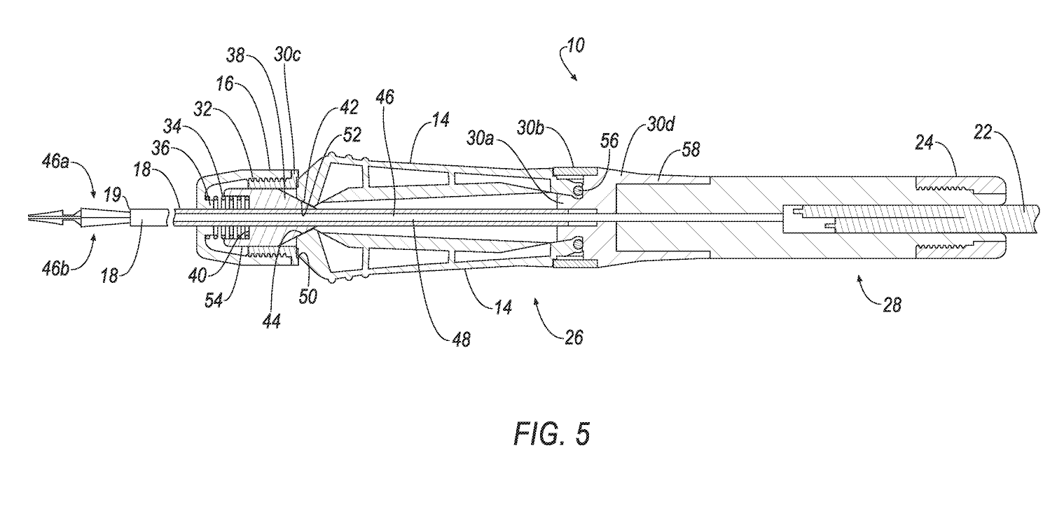

In FIG. 5, the surgeon or other user presses buttons 14 to thereby press chamfered region 52 against beveled face 42 of sheath bushing 38. This action presses sheath bushing 38 against spring 34, thereby compressing spring 34 and moving sheath bushing 38 to the left in the figure. As sheath 18 is connected to sheath bushing 38, the movement to the left of sheath bushing 38 likewise moves sheath 18 to the left as well. As shown in FIG. 5, this movement moves sheath 18 to the left, thereby compressing electrodes 46 inside the inner surface of sheath 18 against their naturally expanded configuration. The compression of electrodes 46 against each other causes surfaces 62 to clamp together.

When the surgeon releases pressure on buttons 14, spring 34 presses sheath bushing 38 back toward the right with respect to the figure. This causes beveled face 42 of the sheath bushing 38 to press against chamfered region 52. This pressure thereby pushes buttons 14 back into their unactuated position. Additionally, the movement of sheath bushing 38 moves sheath 18 to the right with respect to the figure, thereby moving sheath 18 to the right and thus allowing electrodes 46a and 46b to expand, uncompressing surfaces 60.

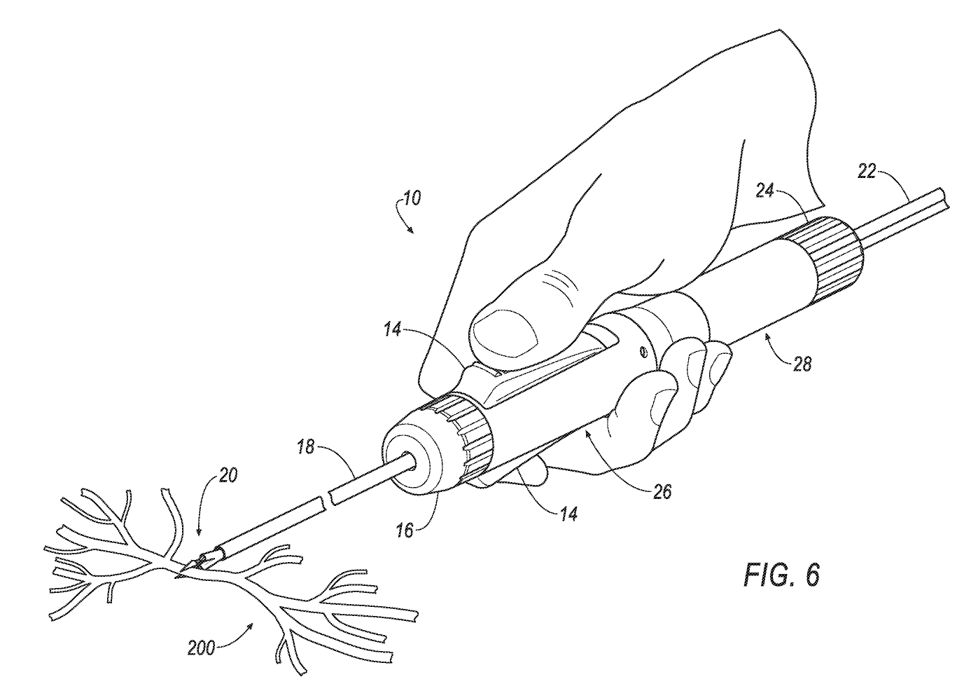

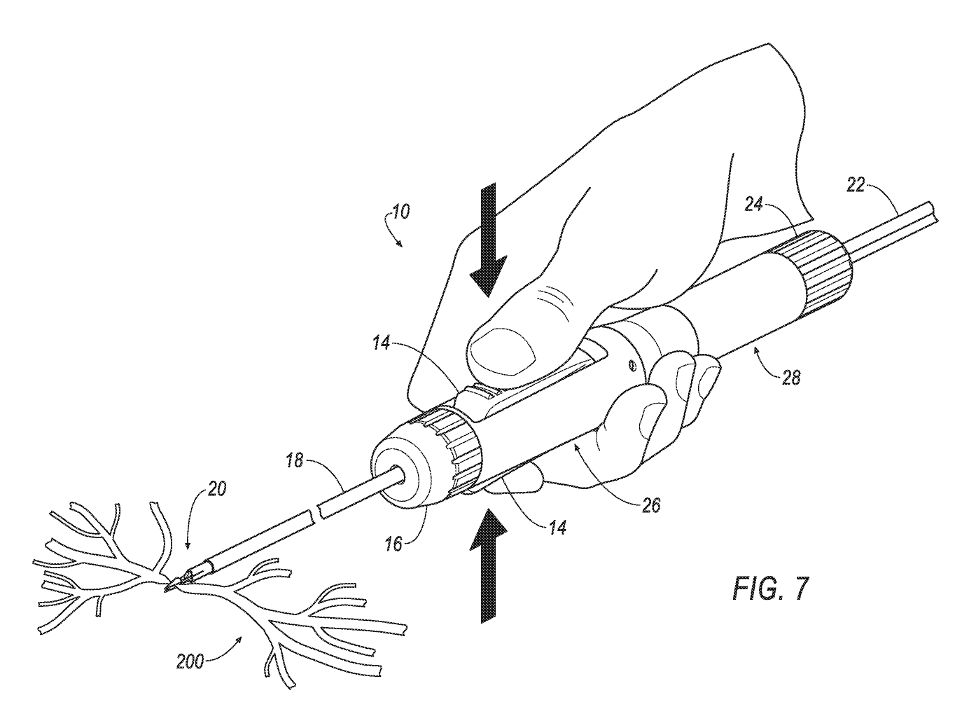

As shown in FIGS. 5 and 6, when the surgeon desires to clamp material during surgery, the surgeon first positions the surfaces 46a and 46b about material 200 and then clamps via use of the electrosurgical forceps by pressing the buttons 14 (as shown in FIG. 6). Thus, the forceps themselves do not axially move and are instead axially fixed. Thereby, only the sheath 18 moves to compress the surfaces 60 against the material 200.

While the invention has been described in connection with preferred embodiments, it will be understood that modifications thereof within the principles outlined above will be evident to those skilled in the art and thus the invention is not limited to the preferred embodiments but is intended to encompass such modifications.

In this specification, various preferred embodiments may have been described with reference to the accompanying drawings. It will, however, be evident that various modifications and changes may be made thereto, and additional embodiments may be implemented, without departing from the broader scope of the invention as set forth in the claims that follow. The present invention is thus not to be interpreted as being limited to particular embodiments and the specification and drawings are to be regarded in an illustrative rather than restrictive sense.

It will be appreciated that the system and methods described herein have broad applications. The foregoing embodiments were chosen and described in order to illustrate principles of the methods and apparatuses as well as some practical applications. The preceding description enables others skilled in the art to utilize methods and apparatuses in various embodiments and with various modifications as are suited to the particular use contemplated. In accordance with the provisions of the patent statutes, the principles and modes of operation of this invention have been explained and illustrated in exemplary embodiments.

It is intended that the scope of the present methods and apparatuses be defined by the following claims. However, it must be understood that this invention may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims. The scope of the invention should be determined, not with reference to the above description, but should instead be determined with reference to the appended claims, along with the full scope of equivalents to which such claims are entitled. It is anticipated and intended that future developments will occur in the arts discussed herein, and that the disclosed systems and methods will be incorporated into such future examples. Furthermore, all terms used in the claims are intended to be given their broadest reasonable constructions and their ordinary meanings as understood by those skilled in the art unless an explicit indication to the contrary is made herein. In particular, use of the singular articles such as “a,” “the,” “said,” etc. should be read to recite one or more of the indicated elements unless a claim recites an explicit limitation to the contrary. It is intended that the following claims define the scope of the invention and that the method and apparatus within the scope of these claims and their equivalents be covered thereby. In sum, it should be understood that the invention is capable of modification and variation and is limited only by the following claims.

What is claimed is:

1. Electrosurgical forceps, comprising:
a body portion;
at least two button extending radially opposite from the body portion, wherein the buttons are movable radially with respect to the body portion;
a first electrode terminating at a first end, wherein the first electrode is connectable to an RF generator to supply electrosurgical current to the first electrode, wherein the first electrode is fixedly positioned in the body portion;
a second electrode positioned parallel to the first electrode, wherein the second electrode is connectable to an RF generator to supply electrosurgical current, wherein the second electrode terminates at a second end, wherein the second electrode is fixedly positioned in the body portion;
a sheath positioned around the first electrode and the second electrode and terminating at a sheath end, wherein the first end and the second end extend axially out from the sheath end, wherein the sheath is movably supported by the body;
an end cap;
a spring located in the end cap; and
a beveled bushing positioned in the end cap and axially pressed against chamfered regions on the first button and the second button, wherein the beveled bushing is rigidly connected to the sheath;
wherein the first end and the second end are spaced when the first button and the second button is un-pressed; and
wherein the sheath moves over the first end and the second end to press the first end against the second end when the buttons are pressed.

2. The electrosurgical forceps according to claim 1, wherein the first end and second end are flat and oppositely facing.

3. The electrosurgical forceps according to claim 1, wherein the first end and the second end are connected to the first electrode and the second electrode by respective connections.

4. The electrosurgical forceps according to claim 3, wherein the connections are a relatively thin region with respect to a thickness of the first electrode and the second electrode.

5. The electrosurgical forceps according to claim 1, wherein the beveled bushing has an aperture disposed axially therein that locates the spring;

6. The electrosurgical forceps according to claim 1, wherein the end cap has a pilot to locate the spring.

7. The electrosurgical forceps according to claim 1, wherein the first electrode and the second electrode is connected to the RF generator.

8. The electrosurgical forceps according to claim 1, wherein the first button and the second button are connected to the body portion by a first hinge and a second hinge respectively.
